(19) United States
(12) Patent Application Publication
(10) Pub. No.: US 2007/0053896 A1
(43) Pub. Date: Mar. 8, 2007

(54) Diagnostic marker for ovarian cancer

(75) Inventors: Nuzhat Ahmed, Yallambie (AU); Gregory Edward Rice, Warranwood (AU); Michael Anthony Quinn, Clifton Hill (AU)

Correspondence Address: JENNIFER M MCCALLUM, PH D, ESQ;THE MCCALLUM LAW FIRM, LLC, 685 BRIGGS STREET, PO BOX 929, ERIE, CO, 80516, US

(73) Assignee: ROYAL WOMEN'S HOSPITAL

(21) Appl. No.: 10/570,751

(22) PCT Filed: Sep. 6, 2004
(86) PCT No.: PCT/AU04/01205

§ 371 Date: Oct. 24, 2006

(30) Foreign Application Priority Data
Sep. 5, 2003 (AU) 2003904844

(51) Int. Cl. A61K 38/00

(52) U.S. Cl. 424/94.5

(57) ABSTRACT

The present invention relates to methods of detecting, monitoring the efficacy of treatment of, and assessing the severity of ovarian cancer, by assessing the concentration of haptoglobin-1 precursor in a sample of biological fluid. The invention also relates to a kit comprising an antibody or nucleic acid probe specific for haptoglobin-1 precursor for use in the diagnosis of ovarian cancer, monitoring the efficacy of treatment of ovarian cancer, or assessing the severity of ovarian cancer.

This application claims priority from Australian provisional application No. 2003904844 dated 5 Sep. 2003.

The present invention relates to methods of diagnosis and monitoring of cancer. In particular, the invention is directed to methods of screening for ovarian cancer and other cancers of the reproductive organs, especially early in the disease, and of monitoring and prognosis for the treatment and clinical management of ovarian cancer and other cancers, and to a molecular marker useful in these methods.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Ovarian cancer is the leading cause of death from gynaecological malignancy and the fourth leading cause of cancer death among Australian women. The cancer is highly metastatic, resulting in secondary growth to distant sites, and the majority of patients diagnosed with advanced epithelial ovarian cancer have widespread metastasis. The dismal outcome for ovarian cancer arises from an inability to detect the tumour at an early, curable stage. As 90% of grade I tumours can be cured by current management methods, patients with ovarian cancer have a good prospect of recovery if diagnosed at an early stage. Currently it is thought that the only practicable way to identify ovarian cancer at an early, curable, stage is to ascertain the identity of proteins which are overexpressed in cancer cells, and hence are secreted from the cancer cells into the peritoneal cavity, and ultimately absorbed into the circulating blood.

To date, no definite marker of ovarian cancer which is suitable for early-stage screening purposes has been identified. CA125 is a serum antigen which is associated with ovarian cancer, and a monoclonal antibody directed against this antigen is widely used in diagnosis and monitoring of the condition. However, CA125 values are not specific indicators of ovarian cancer, as levels of this antigen increase in other gynaecological cancers, non-malignant gynaecological conditions such as ovarian cysts, endometriosis or uterine fibroids, hepatic disease, renal failure, or pancreatitis, and sometimes even in response to infection (Mackay and Creasman, 1995). Moreover, in some cases of ovarian cancer no relationship between CA125 values and disease progression has been identified, making it an unreliable marker for early screening of ovarian cancer. More recently, tumour-associated differentially expressed gene-12 (TADG-12) a serine protease cloned by polymerase chain reaction, has been shown to be overexpressed in approximately 75% of ovarian carcinomas, and has been suggested as an alternative marker (Underwood L J, 2000). However, this marker has potential for false negatives because of its low degree of association with ovarian cancer.

Hence to reduce mortality from ovarian cancer, there is a desperate need to identify molecular markers which are preferably detectable in blood, plasma or serum to complement the use of existing tests in detecting early-stage disease.

Proteomics is an emerging technology which can identify protein molecules in a high-throughput discovery approach in patient's serum, other biological fluids and tissues, providing information about proteins which are secreted or released from tumour cells at sufficient concentrations. The serum proteins of the cancer patient represent a rich source of biomarkers, due to the modification of the serum protein profile with disease progression. As serum circulates through the diseased organ it picks up proteins produced by the tumour and its host microenvironment. Hence a cancer-related serum proteome represents proteins which are over-expressed or abnormally shed as a result of the disease process, or are representative of proteins which are removed from the proteome as a result of abnormal activation of proteolytic degradation pathways. Thus a small but significant change in protein molecules specific to cancer cells, even at the earliest stage of the disease, may be reflected in the serum proteome, enabling one to identify combinations of biomarkers which would be more effective in detecting and monitoring the disease. Over-expressed proteins which are secreted from cancer cells and are absorbed by circulating blood are potential candidate markers for use in assays measuring the protein product in serum. Secreted proteins may evoke antibody responses in the patient, in which case an antibody-based serum marker may be feasible.

The technologies of electrospray ionisation mass spectrometry, matrix-assisted laser desorption ionization time-of-flight mass spectrometry (MALDI-TOFMS) and surface-enhanced laser desorption ionization time-of-flight mass spectrometry (SELDI-TOFMS), which are commonly used in proteomic methods, have the potential to identify patterns or changes in thousands of proteins, and enable global analysis of almost all the small molecular weight proteins present in complex biological fluids such as serum or plasma.

A serological proteomic pattern of ovarian cancer patients which discriminates cancerous from non-cancerous groups with a positive predictive value of 94% has recently been described (Petricoin et al, 2002). This approach represents a novel direction in the search for biomarker discovery for early stage screening of ovarian cancer, in which a distinct profile of proteins from early-stage cancer patients can create a discriminatory pattern of proteins relative to that of normal subjects which can be used as a diagnostic standard. However, the applicability of this approach is still being evaluated, because of its low specificity.

Haptoglobin is an acute phase glycoprotein which binds haemoglobin, thus preventing iron loss and renal damage as a consequence of inflammation or injury (Wassell, 2000). The native form of mature haptoglobin is a tetramer of molecular weight approximately 90,000 kDa, composed of two non-identical α and β-subunits linked by intermolecular disulfide bonds (Hanley and Heath, 2000). Haptoglobin has three major phenotypic forms, haptoglobin 1-1, haptoglobin 2-1 and haptoglobin 2-2, and either or all alleles may be present in a single individual. Individual phenotypes are associated with a variety of conditions, including cardiovascular and autoimmune disorders and some malignant conditions. However, in other cancers, such as prostate cancer, haptogloblin expression is abolished as a result of malignant transformation (Meechan et al, 2002).

In vivo, haptoglobin is synthesized as a single polypeptide precursor exhibiting a molecular weight of 38,000 kDa. It is thought that all three phenotypes of the mature protein are derived from a single precursor, haptoglobin-1 precursor. The polypeptide precursor is proteolytically processed to form the α and β-subunits of the native protein (Haugen et al, 1981). The precursor protein includes an amino-terminal 18 residue signal sequence before the a chain, and/or an intervening polypeptide between the α and β-regions (Misumi et al, 1983). In vivo, post-translational events result in the proteolytic removal of the signal sequence and the incorporation of the core oligosaccharide side chains into the β-region by membrane-associated enzyme systems (Haugen et al, 1981). Post-translational modification may also result in the cleavage of both α and β regions of the precursor polypeptide to form the native protein (Haugen et al, 1981). The biological implications of the unique mode of biosynthesis and processing of haptoglobin are still not clear, but it has been shown that a substantial proportion of the newly-synthesized haptoglobin is secreted as a single polypeptide precursor (Misumi et al, 1983).

Elevated concentrations of serum haptoglobin were first reported in ovarian cancer patients in the early 1970s. The haptoglobin concentration was shown to be affected by the degree of tumour burden, and was not dependent on the histological type or grade of ovarian malignancy (Mueller et al, 1971). Some studies have shown increased fucosylation and other glycosylation changes in the serum haptoglobin of ovarian cancer patients (Thompson et al, 1992). Recently, a correlation between levels of haptoglobin, CA 125 and interleukin-6 has been shown in ovarian cancer (Dobryszycka et al, 1999). As these studies relied on spectrophotometric (Mueller et al, 1971), immunodiffusion (Thompson et al, 1992) and electrophoretic (Thompson et al, 1992) detection of haptoglobin, the homologous native protein was detected rather than the haptoglobin precursor.

Ono et al, 2000 discloses the expression of mRNA corresponding to haptoglobin α(15)-β precursor in ovarian tumour tissues. These authors did not suggest that haptoglobin-1 precursor could be detected in serum and ascites fluid of ovarian cancer patients. Although it is known that expression of mature haptoglobin in biological fluids is up-regulated in conditions such as cancer, arthritis, and proteinuria, the precursor form of haptoglobin has not been detected in these conditions.

None of these previous reports has suggested that detection or measurement of any haptoglobin precursor in a biological fluid might be useful in the diagnosis, staging or prognosis of ovarian cancer or any other cancers.

### SUMMARY OF THE INVENTION

Using proteomic and Western blotting approaches we have now identified haptoglobin-1 precursor in the serum of early stage ovarian cancer patients. We have shown that the haptoglobin-1 precursor concentration is elevated in the serum of ovarian cancer patients compared to normal. Thus we propose haptoglobin-l precursor as a candidate for development as a biomarker. Moreover, our finding that haptoglobin-1 precursor expression increases with the progression of ovarian cancer makes it an ideal candidate to complement or replace the widely-used but non-specific CA125 marker.

In a first aspect, the invention provides a method of detection of ovarian cancer, comprising the step of determining the concentration of haptoglobin-1 precursor in a sample of a biological fluid from a subject suspected to be suffering from ovarian cancer, wherein an increased concentration of haptoglobin-1 precursor compared to the concentration of haptoglobin-1 precursor in a control sample is an indication of the presence of the cancer.

The ability to use a sample of biological fluid to detect haptoglobin-1 precursor provides relative ease in obtaining samples compared with obtaining a tissue sample, such as a biopsy. Moreover, it enables the haptoglobin-1 precursor to be detected earlier in the development of an ovarian cancer, as biopsy samples are often taken late in the progression of a disease. In the case of ovarian cancer, a biopsy is frequently not taken until the tumour is surgically resected.

In a second aspect, the invention provides a method of monitoring the efficacy of treatment of ovarian cancer, comprising the step of determining the concentration of haptoglobin-l precursor in a sample of a biological fluid from a subject suspected to be suffering from ovarian cancer, wherein a decrease in haptoglobin-1 precursor level compared to the level before treatment is an indication of efficacy of the treatment.

In a third aspect, the invention provides a method of assessing the severity of ovarian cancer, comprising the step of quantitatively determining the concentration of haptoglobin-1 precursor in a biological fluid of a subject diagnosed with, or suspected to be suffering from, ovarian cancer, wherein an increased concentration of haptoglobin-1 precursor compared to the concentration of haptoglobin-1 precursor in a control sample is an indication of the presence and/or severity of the cancer. In all three approaches of this invention the levels of haptoglobin-1 precursor may optionally be correlated with one or more other markers of ovarian cancer.

In all three aspects of the invention the biological fluid may be blood, plasma, serum, ascitic fluid or urine. The person skilled in the art will readily be able to determine whether other biological fluids, such as saliva, could also be used.

The concentrations of haptoglobin-1 precursor may be determined by any convenient method for detecting either the haptoglobin-1 precursor protein or the nucleic acid encoding it, including but not limited to ELISA, radioimmunoassay, chemiluminescence assay, realtime PCR, nucleic acid hybridization methods and the like. Specific antibodies, including monoclonal antibodies, directed against haptoglobin-1 precursor can readily be prepared using conventional techniques, and may be used in such methods. Preferably these antibodies do not react with epitopes within the a chain. The concentration may be determined qualitatively or quantitatively.

The sample of biological fluid may optionally be subjected to a preliminary step to delete high abundance proteins such as albumin, using Affi-Gel Blue Protein A or Blue Sepharose-Protein A columns, or using methods described in International patent application No. PCT/AU03/01075 filed in the name of Royal Women's Hospital on 22 Aug. 2003, corresponding to Australian provisional patent application No. 2002951240 filed on 23 Aug. 2002. This increases the sensitivity of detection of low abundance proteins.

Optionally the methods of the invention also comprise the step of determining levels of another ovarian cancer marker, such as integrin-linked kinase (ILK), CA125, TADG-12, mesothelin, kallikrein 10, prostasin, osteopontin, creatine kinase β, serotransferrin, neutrophil-gelatinase associated lipocalin (NGAL), CD163, or Gc-globulin. Alternatively, elevated expression of one or more other putative markers of ovarian cancer, such as mesothelin, kallikrein 10, prostasin, osteopontin, or creatine kinase β, may also be detected. The second marker may be detected at the DNA, RNA or protein level, using methods known in the art.

In a fourth aspect, the invention provides a kit for use in:
a) diagnosis of ovarian cancer;
b) monitoring of the efficacy of treatment of ovarian cancer; or
c) assessment of the severity of ovarian cancer;
   comprising an antibody or a nucleic acid probe specific for haptoglobin-1 precursor.

In a fifth aspect, the invention provides the use of an antibody or a nucleic acid probe specific for haptoglobin-1 precursor in:
a) diagnosis of ovarian cancer;
b) monitoring the efficacy of treatment of ovarian cancer; or
c) assessing the severity of ovarian cancer.

In both the fourth and fifth aspects of the invention the antibody is preferably a monoclonal antibody. More preferably the antibody does not react with an epitope within the a chain, and even more preferably the antibody is specific for haptoglobin-1 precurser.

While it is particularly contemplated that the present invention is suitable for use in humans, it is also applicable to veterinary use, including use in companion animals such as dogs and cats, and domestic animals such as horses, cattle and sheep, or zoo animals such as non-human primates, felids, canids, bovids, and ungulates.

### BRIEF DESCRIPTION OF THE FIGURES

FIG. 1 shows the result of pretreatment of serum samples with Affi-Gel Blue and protein A prior to two-dimensional electrophoresis (2-DE), illustrating depletion of albumin and enhanced detection of low abundance proteins. FIG. 1a: two-dimensional electrophoresis profile of normal serum visualized by staining with SYPRO Ruby; FIG. 1b: 2-DE profile of serum pretreated with Affi-Gel Blue and protein.

FIG. 2 shows the results of two-dimensional electrophoresis, illustrating enhanced expression of six different isoforms of haptoglobin-1 precursor in the serum of ovarian cancer patients compared to that of normal subjects as identified by proteomic analysis. FIG. 2a: Normal subjects; FIG. 2b: grade 1 ovarian cancer patients; FIG. 2c: grade 2 ovarian cancer patients and FIG. 2d: grade 3 ovarian cancer patients.

FIG. 3 shows two-dimensional electrophoresis profiles demonstrating haptoglobin-1 precursor expression in ascitic fluid (AS) from ovarian cancer patients.

FIG. 4 shows two-dimensional electrophoresis profiles of ovarian cancer patients of different grades, demonstrating differential expression of proteins. FIG. 4a: grade 1; FIG. 4b: grade 2; FIG. 4c: grade 3.

FIG. 5 shows the results of MALDI-TOF MS and n-ESIQ(q)TOF MS mass fingerprinting analysis of the six proteins isolated form the 2-DE gels.

FIG. 6a illustrates the levels of immunoreactive 38 kDa haptoglobin-l precursor in the serum of grade 1 and grade 3 ovarian cancer patients, as determined by one-dimensional electrophoresis and Western blot using monoclonal anti-haptoglobin antibody.

FIG. 6b illustrates the levels of immunoreactive 38 kDa haptoglobin-1 precursor in the serum of normal, benign, and boarderline subjects, and grade 1, grade 2 and grade 3 ovarian cancer patients, as determined by one-dimensional electrophoresis and Western blot using monoclonal anti-haptoglobin antibody.

FIG. 6c shows the levels of haptoglobin-1 precursor expression in serum of normal, benign and borderline subjects, and grade 1, 2 and 3 ovarian cancer patients.

FIGS. 7a, 7b and 7c show elevated levels of immunoreactive haptoglobin-1 precursor isoforms in the serum of (a) normal subject, (b) grade 1 and (c) grade 3 ovarian cancer patients, compared to levels in normal serum. The level of expression was determined by two dimensional gel electrophoresis and Western blotting using monoclonal anti-haptoglobin antibody.

FIG. 8 shows the results of immunohistochemical detection of immunoreactive haptoglobin-1 precursor in tissue samples, using a monoclonal antibody against haptoglobin. Immunoreactive haptoglobin-1 precursor was absent from normal ovaries (panel a), but was present in grade 1, 2 and 3 ovarian tumour tissues (serous tumour, panel b and endometrioid tumour; panels c and d).

FIG. 9a illustrates the haptoglobin-1 precursor expression profile in a sample from a grade 3 ovarian cancer patient, before and after chemotherapy treatment, as measured by two-dimensional electrophoresis.

FIG. 9b shows the relative levels of expression of the different isoforms of haptoglobin-1 precursor in a sample from an ovarian cancer patient, before and after chemotherapy treatment.

### DETAILED DESCRIPTION OF THE INVENTION

Definitions

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

As used herein, the singular forms “a”, “an”, and “the” include the corresponding plural reference unless the context clearly dictates otherwise. Thus, for example, a reference to “a protein” includes a plurality of such proteins, and a reference to “a molecule” is a reference to one or more molecules. Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

When a range of values is expressed, it will be clearly understood that this range encompasses the upper and lower limits of the range, and all values in between those limits.

“Haptoglobin-1” refers to the mature glycolysated tetramer of molecular weight approximately 90 kD.

“Haptoglobin-1 precursor” refers to the single chain precursor protein of molecular weight approximately 38 kD, which includes the 18 amino acid signal sequence.

“Immunoreactive haptoglobin-1 precursor” refers to haptoglobin-1 precursor detected using monoclonal antibody directed to mature haptoglobin-1.

Abbreviations used herein are as follows:
1-DE one -dimensional electrophoresis
2-DE two-dimensional electrophoresis
ir immunoreactive
MALDI-TOF matrix-assisted laser desorption interferometry-time of flight
MS mass spectroscopy
MS/MS tandem mass spectroscopy
SELDI-TOF MS surface-enhanced laser desorption
ionization time-of-flight mass spectrometry
TOF MS time of flight mass spectrometry

We have found that haptoglobin precursor is present in ascites and in the serum of grade 1, grade 2 and grade 3 ovarian cancer patients. In ovarian cancer tissues, immunoreactive haptoglobin-1 precursor is present in the epithelial cells, stroma and ovarian vessels. Haptoglobin-1 precursor is >90% homologous to mature haptoglobin. Hence a monoclonal antibody against haptoglobin is able to detect immunoreactive haptoglobin-1 precursor.

These data are consistent with the hypothesis that overexpression of haptoglobin-1 precursor is an early event in the onset of ovarian cancer. Thus enhanced expression of haptoglobin-1 precursor may represent a condition of acute response, which is a pre-requisite for tumour progression.

Without wishing to be limited to any proposed mechanism, we believe that since most secretory proteins are initially synthesized as larger precursors with an extended NH₂-terminal sequence which is cleaved at the late stage of the secretory process, either within the Golgi complex or in related vesicles, the elevated levels of the haptoglobin-1 precursor in the serum of cancer patients may result from defective intracellular processing which is specific to cancer cells.

Transcriptional profiling, or the related serial analysis of gene expression, subtractive hybridization and differential display technologies, has identified fourteen candidate ovarian cancer markers (Mok et al, 2001; Schummer et al, 1999). Among these proteins, mesothelin and kallikrein 10 were identified by the monoclonal antibody and candidate gene approaches, respectively (Scholler et al, 1999; Luo et al, 2001). Mesothelin is elevated in the serum of 76% of ovarian cancer patients (Diamandis et al, 2000), and kallikrein 10 is elevated in 56% of ovarian cancer patients (Luo et al, 2001). It has been suggested that tests for mesothelin and/or kallikrein 10 may be able to complement the CA125 test, increasing the prospect of detecting ovarian cancer at an early, curable stage.

Gene array technology has been used to identify prostasin, a serine protease, previously identified in prostatic secretions, osteopontin, a secreted bone morphogen, and creatine kinase B, a marker for renal and lung cancers, as being elevated in serum from patients with ovarian cancer (Mok et al, 2001; Kim et al, 2002). These data indicate that screening approaches at the DNA or RNA level may be able to identify a series of markers which also have the potential to complement the currently used CA125 test and to provide increased specificity and sensitivity.

We have recently identified a cell-free immunoreactive form of integrin-linked kinase (ILK) as a marker for ovarian cancer which is highly correlated with the stage of the cancer. See International patent application No. PCT/AU03/01058 in the name of The Royal Women's Hospital, filed on 20 August 2003.

One or more additional markers for ovarian cancer, such as ILK (PCT/AU03/01058), CA125 (Mackay and Creasman, 1995), TADG-12 (Underwood L J, 2000), or the more recently-described markers, serotransferrin (Kawakami et al, 1999), neutrophil gelatinase associated lipocalin (Kjeldsen et al, 1994>, soluble CD163 (Baeton et al, 2003) and Gc-globulin (Jorgensen et al, 2004) may be used in the methods of the invention as an adjunct to the detection of haptoglobin-1 precursor.

General Methods

Two-dimensional electrophoresis and mass spectrometry First Dimension Separation: Twenty five μg of serum protein were mixed with rehydration buffer (7 M urea, 2 M thiourea, 100 mM dithiothreitol (DTT), 4% (3-[(3-cholamidopropyl)dimethylammonio]-1-propanesulfonate) (CHAPS), 0.5% carrier ampholytes, 0.01% bromophenol blue (BPB) 40 mM Tris and pH 4-7) to a final volume of 200 μl, and incubated for 1 h at room temperature. This mixture was then applied to a Ready Strip (11 cm, pH 4-7; Bio-Rad Laboratories, USA) and actively rehydrated at 50 V at 20° C. for 16 h. Serum proteins were subjected to isoelectric focusing at 250 V for 15 min; the voltage was then slowly ramped up to 8000 V for 150 min, and then maintained at 8000 V for a total of 35000 Vh/gel (i.e. a total of 42000 Vh per gel). The Ready Strips were then stored at −80° C. prior to second dimension separation.

Second Dimension Separation: Ready Strips from the first dimension separation were equilibrated in 5 ml of equilibration buffer (50 mM Tris-HCl pH 8.8, 6 M urea, 30% glycerol, 2% sodium dodecyl sulfate (SDS), 0.01% BPB, 2 mM tributyl phosphine (TBP)). Strips were rinsed in Tris-glycine-SDS running buffer (25 mM Tris, 192 mM glycine, 0.1% w/v SDS; pH 8.3) and then applied to the top of a 10% Tris-HCl Precast Criterion Gel (Bio-Rad Laboratories, USA). Low melting point agarose (0.5% in running buffer containing BPB) was layered on top of the strip. Molecular weight markers (150, 100, 75, 50, 37 and 25 kDa) were run simultaneously. Gels were electrophoresed at 10 mA/gel for 1 h, 20 mA/gel for 2 h and then 30 mA/gel for 30 min. Gels were then fixed in methanol/acetic acid (40%/10% in dH₂O) for 1 h at room temperature and incubated in SYPRO Ruby® (Bio-Rad laboratories, USA) for 16 h at room temperature on a rocking platform. Gels were de-stained for 1 h in methanol/acetic acid (10%/7% in dH20), imaged using a Bio-Rad FX imager at 100 nm resolution, and analyzed using PDQuest version 6 software (Bio-Rad laboratories, USA). The computer program identified protein spots from the digitized images of the gel. Analyses of some serum samples were repeated three times to assess the variability between the experiments on different gels.

Mass spectrometry: Following the imaging, the gels were stained with Coomassie blue to enable visual identification and isolation of the protein bonds. Coomassie stained bands were excised from the gel and digested with trypsin. Mass spectrometry experiments were performed on an Ettan MALDI-TOF instrument (Amersham Bioscience, UK) and API QSTAR Pulsar i Mass spectrometer (Applied Biosystems, MDS Sciex, Framingham, USA). TOFMS data were searched via PepSea Server, which is included in the Analyst Software (Applied Biosystems, MDS Sciex, Framingham, USA). Tandem MS data were searched via the MASCOT search engine.

The invention will now be described in detail by way of reference only to the following non-limiting examples and drawings.

### EXAMPLE 1

### Removal of High Abundant Albumin from Human Serum

Human serum samples were treated with a mixture of Affigel-Blue and protein A (5:1) in the form of a spin column (Bio-Rad Laboratories, USA). The spin columns contained a mixture of Affi-Gel Blue and Protein A, which selectively binds and removes albumin and immunoglobulin. The spin columns were washed twice with 1 ml of binding buffer (20 mM phosphate buffer, pH 7.0) by centrifugation for 20 sec at 1000× g. 50 μl of serum was added to 150 μl of binding buffer, mixed by vortexing, and loaded on the spin columns. Following incubation at room temperature for 1 h, columns were centrifuged for 20 sec at 1000× g to collect the eluate. The columns were washed with 200 μl of binding buffer and combined with the first eluate to form the depleted serum sample. The total protein concentration of the combined eluate was determined. The eluate was stored at −80° C. until further analysis.

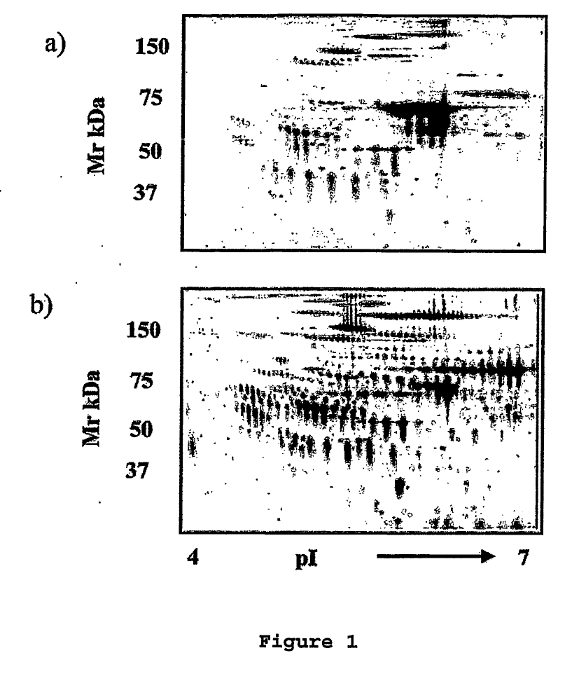

FIG. 1a demonstrates a typical 2-DE human serum profile visualized by SYPRO Ruby staining. More than 300 proteins were detected and localized between pI 4-7 and molecular mass range of 20-200 kDa. The albumin smear at around 68 kDa was present in untreated serum, but within 1 h of Affi-Gel Blue and protein A treatment significant loss of albumin was achieved, with no significant loss of other proteins displayed. Concomitant with the removal of albumin there was a significant enhancement in the staining intensity of several protein spots, as shown in FIG. 1b. These results indicate that Affi-Gel Blue and protein A treatment of human serum results in the removal of high abundance albumin, thereby increasing the detection of low abundance proteins which would have remained obscured in the presence of albumin. We have implemented this approach of albumin clearance for the identification of differentially-expressed low abundance proteins in the serum of ovarian cancer patients.

### EXAMPLE 2

### Expression of Haptoglobin-1 Precursor in Serum and Ascites from Ovarian Cancer Patients

Proteomic analysis and mass spectrometry were used to evaluate the expression of haptoglobin-1 precursor in the serum of normal healthy women and of ovarian cancer patients. Cancer patients were graded according to standard histological methods (Silverberg, 2000).

The mean age of women in the control group and women with ovarian cancer was 47 and 62 years respectively. Whole blood (10 ml) was collected by venepuncture into plain collection tubes for serum (blood was allowed to clot at room temperature for 30 min). Samples were centrifuged at 2000 g for 10 min after which serum was collected. An aliquot (100 μl) was removed for the determination of total protein. Serum was stored at 80° C. until analyzed.

Serum samples from 8 normal female subjects and 19 ovarian cancer patients were analysed for the expression of haptoglobin-1 precursor. Of the patients, 6 were grade 1, 8 were grade 2, and 24 were grade 3. Ascitic fluid of ovarian cancer patients was also tested for haptoglobin-1 precursor expression. Haptoglobin-1 precursor expression was detected in serum and ascitic fluid by proteomic analysis and by Western blotting under non-reducing conditions, using a monoclonal antibody against mature haptoglobin (Sigma, St Louis, USA). Haptoglobin-1 precursor is >90% homologous to mature haptoglobin. Hence the monoclonal antibody against haptoglobin is able to detect immunoreactive haptoglobin-1 precursor.

Whole blood (2 ml) was collected by venepuncture into plain collection tubes, and allowed to clot at room temperature for 30 min. Samples were then centrifuged at 2000 g for 10 min, after which serum was collected. An aliquot (100 μl) was removed for the determination of total protein. Serum was stored at −80° C. until analysed. Blood specimens were thawed at room temperature, and two-dimensional electrophoresis was performed on the specimens.

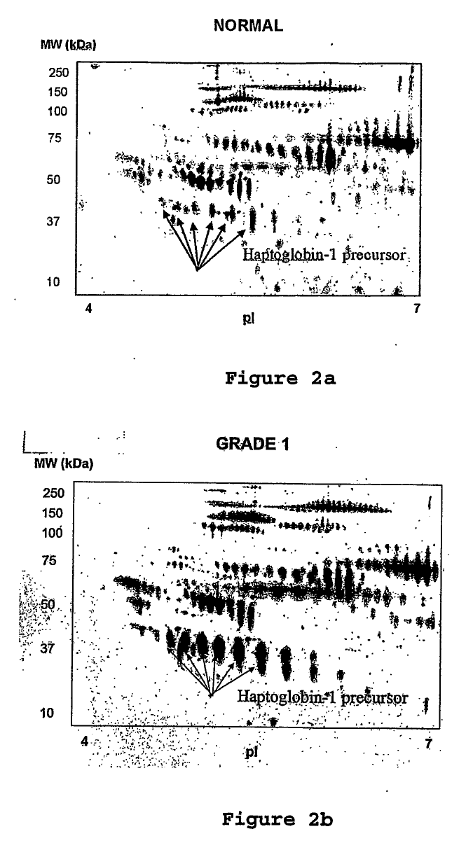

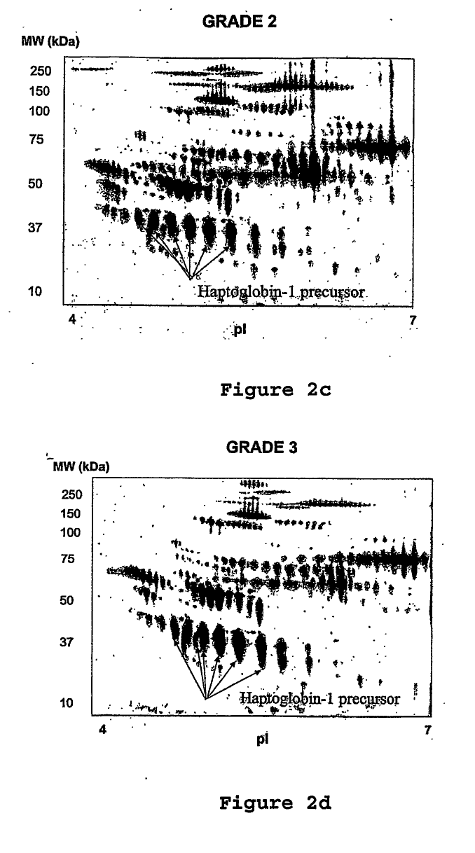

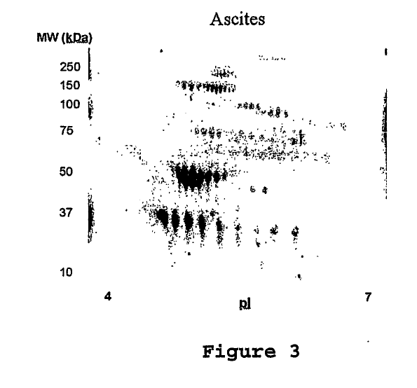

FIG. 2 shows the results of proteomic analysis of expression of haptoglobin-1 precursor in the serum of normal subjects and in grade 1, grade 2 and grade 3 ovarian cancer patients. FIG. 3 shows the results of proteomic analysis of expression of haptoglobin-1 precursor in ascitic fluid from ovarian cancer patients.

### EXAMPLE 3

### Serum Protein Profile of Ovarian Cancer Patients at Different Histological Grades

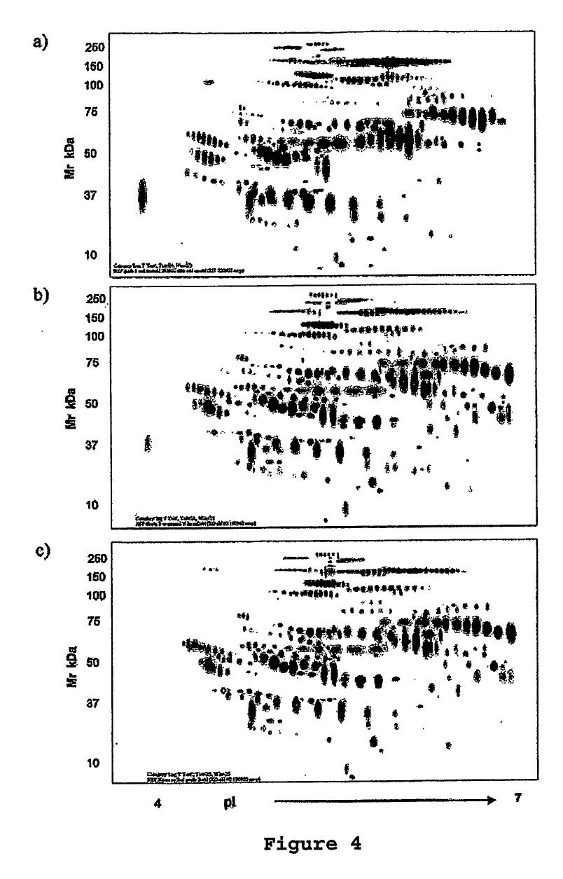

Protein profiles of the serum of grade 1 (n=6), grade 2 (n=8) and grade 3 (n=24) ovarian cancer patients were analyzed by 2-DE and visualized by staining with SYPRO-Ruby. Protein profiles of replicate sets of samples from cancer patients were compared with the serum of normal healthy women (n=8) using PDQuest software, and the Gaussian profiles are shown in FIG. 4. The quantitative evaluation of the differentially expressed serum proteins in normal vs grade 1, grade 2 or grade 3 ovarian cancer patients was performed using Student's t-test. Significant differences in the overall profiles of serum proteins were obtained in grade 1, 2 and 3 ovarian cancer patients compared to normal healthy volunteers. Compared to normal serum, twenty-four proteins were differentially expressed in the serum of grade 1 ovarian cancer patients (FIG. 4a). Of these proteins, fifteen proteins were up-regulated by two-fold, four proteins by five-fold and two proteins by ten-fold. In contrast, one protein was down-regulated by two, five and ten-fold respectively. In grade 2 cancer patients, differential expression of thirty-one proteins was observed of which twenty-five were up-regulated by two-fold, four by five-fold and two by ten-fold. Analysis of serum from grade 3 cancer patients demonstrated two-fold down-regulation of thirteen proteins out of twenty-five differentially-expressed proteins (FIGS. 4b and 4c). Six proteins were up-regulated by two-fold, three by five-fold and two by ten-fold respectively (p<0.05).

Some proteins were found to be uniquely expressed only in the serum of a specific pathological grade of cancer patients, and some proteins were not consistently expressed among the three histological grades of cancer patients. To ensure consistency in the observed differential expression profile, analyses of serum samples from the same patient prepared on three different days were repeated three times, and investigated to eliminate confounding factors that may arise from sample handling. No substantial variation in the profile of protein spots of the same sample repeated on different days was detected.

Among the differentially-expressed serum proteins, ten proteins were found to be consistently expressed in grade 1, 2 and 3 cancer patients (p>0.05). Six of these proteins, which had approximate molecular weights of 40 kDa and pI of 5.9-6.6, were significantly over-expressed in the serum of grade 1, 2 and 3 ovarian cancer patients. These were selected for further analysis and identification.

### EXAMPLE 4

### Identification of Proteins Over-Expressed in Ovarian Cancer Patients

The six proteins found in Example 3 to be over-expressed in ovarian cancer patients were identified by nano-electrospray quadrupole quadrupole time of flight mass spectrometry (ESIQ(q)TOF MS) and matrix-assisted laser desorption ionization time of flight mass spectrometry (MALDI-TOF MS) analysis.

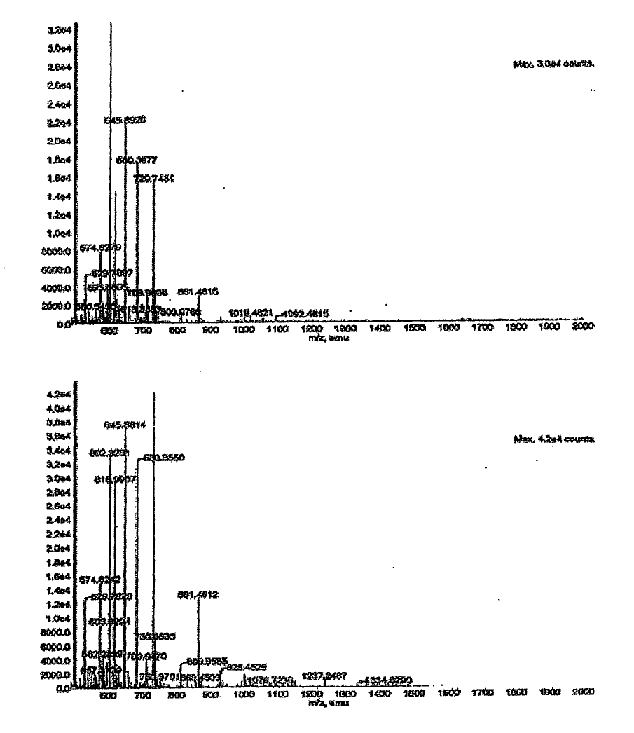

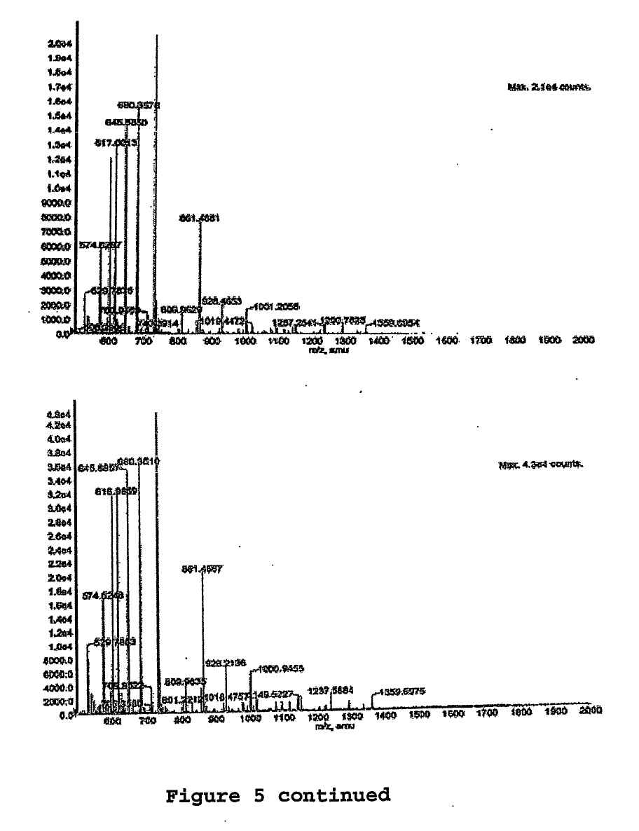

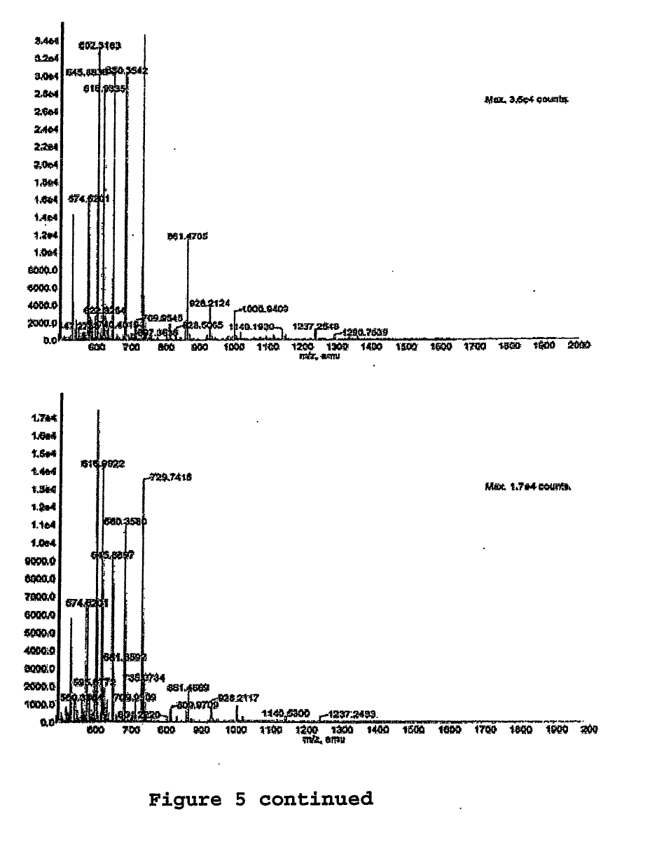

Mass fingerprinting spectra from the six proteins, shown in FIG. 5, showed identical fragmentation patterns of the peptides, suggesting the possibility of a series of post-translational modifications of a single protein separating at different pI and/or molecular mass values. MS/MS analysis of the six proteins confirmed their identity as isoforms of haptoglobin-1 precursor (Swissprot accession number P00737), a protein with a molecular mass of 38.42 kDa and pI of 6.1-6.6 which shares 90% homology to the liver glycoprotein haptoglobin, which is present in normal serum (Beutler et al, 2002). The amino acid sequences of these peptides are summarized in Table 1. The peptide sequence obtained encompassed amino acid sequences corresponding to different regions of haptoglobin-1 precursor.

TABLE 1

Peptide sequences of spots 1-6 in the serum
of grade 3 ovarian cancer patient

| | |
|---|---|
| Peptide Sequences | Amino acid position |

| | |
|---|---|
| ILGGHLDAK | 103-111 |
| DIAPTLTLYVGK | 157-168 |
| RVMPICLPSKDYAEVGRV | 202-219 |
| YVMLPVADQDQCIRH | 239-253 |
| SPVGVQPILNERTFCAGMSK | 266-286 |
| YQEDTCYGDAGSAFAVHDLE | 287-320 |
| EDTWYATGILSFDK | |

Blast Search Result
Swiss Prot Accession number: P00737
Mass: 38,427 D
Protein identified: Human Haptoglobin-1 precursor

The locations of these peptides in the full-length haptoglobin-1 precursor sequence are indicated in Table 2.

TABLE 2

Peptides (underlined) from spots 1-6 corresponding to
haptoglobin-1 precursor molecule:

| | |
|---|---|
| MSALGAVIALLLWGQLFAVDSGNDVTDIADDGCPKPPEIA | 40 |
| HGYVEHSVRYQCKNYYKLRTEGDGVYTLNNEKQWINKAVG | 80 |
| DKLPECEAVCGKPNPANPVQR¹⁰³ILGGHLDAK¹¹¹GSFPWQAKM | 120 |
| VSMMNLTTGATLINEQWLLTTAKNLFLNHSENATAK¹⁵⁷DIAP | 160 |
| TLTLYVGK¹⁶⁸KQLVEIEKVVLHPNYSQVDIGLIKLNQKVSVN | 200 |
| E²⁰²RVMPICLPSKDYAEVGRV²¹⁹GYVSGWGRNANFKFTDHLK²³⁹YV | 240 |
| MLPVADQDQCIRH²⁵³YEGSTVPEKKTPK²⁶⁶SPVGVQPILNEHTF | 280 |
| CRGMSK²⁸⁶ ²⁸⁷YQEDTCYGDAAGSAFAVHDLEEDTWYATGILSFDK³²⁰ | 320 |
| SCAVAEYGVYVKVTSIQDWVQKTIAEN | |

Differences in the glycosylation pattern of the protein have the potential to change both the pI and molecular mass of the protein, and different sialylated forms of haptoglobin have been demonstrated in normal serum using the 2-DE approach (Wilson et al, 2002). Our results, which demonstrate that six different isoforms of haptoglobin-l precursor are present in the serum of ovarian cancer patients, are consistent with these observations.

Further confirmation that these proteins were isoforms of haptoglobin-1 precursor was obtained by Western blotting using monoclonal anti-human haptoglobin antibody. The six isoforms of haptoglobin-1 precursor were detected by 1-DE or 2-DE and Western blotting as a chain of protein spots with slightly different molecular masses and different pIs. The native protein has 90% homology to the precursor, so monoclonal-anti-haptoglobin antibody is expected to visualize haptoglobin isoform precursors on 2-DE Western blot.

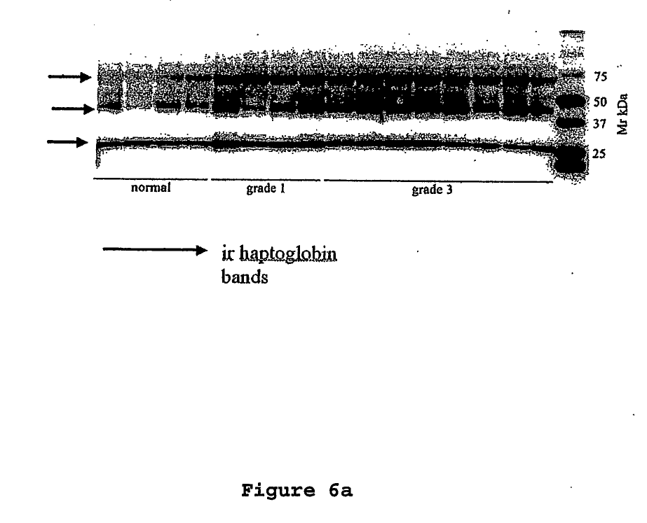

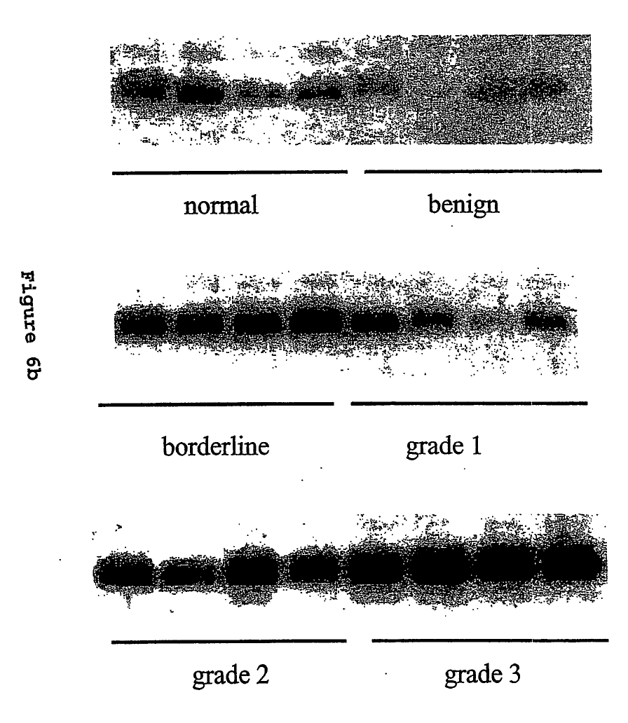

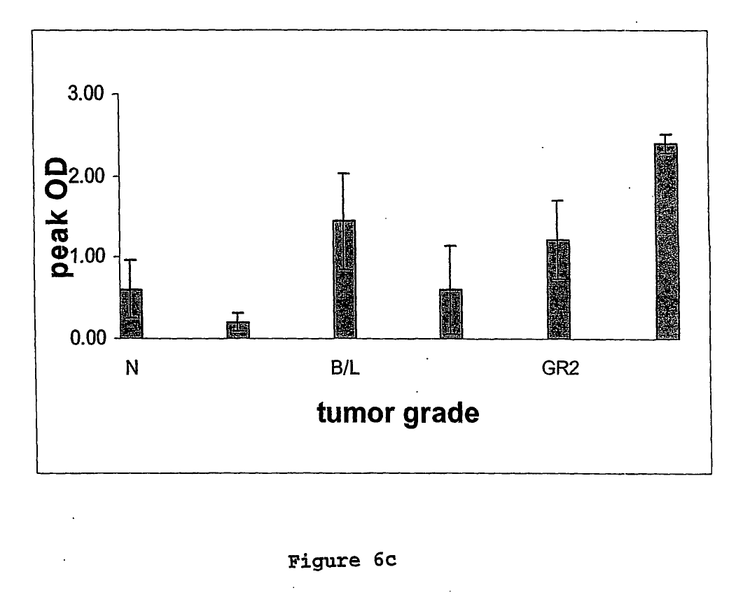

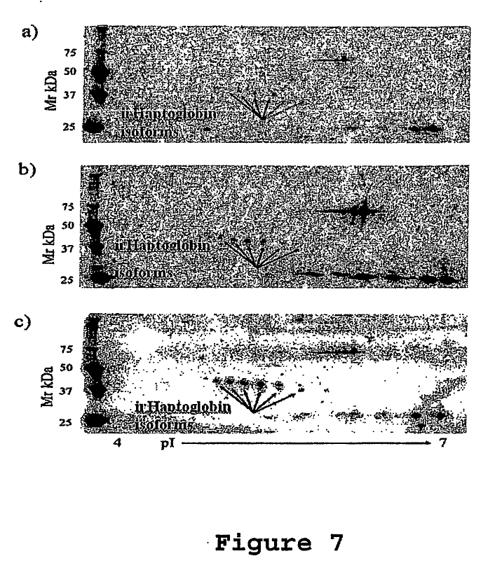

For Western blots, serum samples were incubated with 5 volumes of Laemmli buffer. Specimens containing equal amounts of protein (60 μg) were electrophoresed on a 10% SDS-PAGE gels under non-reducing conditions, and then transferred to nitrocellulose membranes. Membranes were probed with primary haptoglobin antibody (Sigma, USA) followed by peroxidase-labelled secondary antibody (Amersham, UK) and visualised by the ECL detection system (Amersham, UK) according to the manufacturer's instructions. The results are shown in FIGS. 6 and 7.

FIG. 6a shows the 1-DE Western profile of haptoglobin molecules in the serum of healthy volunteers and ovarian cancer patients. The antibody predominantly recognizes three bands at approximate molecular weights 20, 40 and 70 kDa. Interestingly, the expression of the proteins identified by anti-haptoglobin antibody at 40 and 70 kDa was greater in grade 1 and 3 cancer patients than in healthy adults. Consistent with this, high reactivity was observed with the set of six proteins at 40 kDa molecular weight by 2-DE Western blot (FIGS. 6b, 6c and 6d). Similar results were obtained using 2-DE and Western blotting, as illustrated in FIG. 7. Reactivity was also observed with two additional proteins at molecular weight 20 and 70 kDa, and was consistent with the 1-DE profile. The identity of the proteins at molecular weights 20 and 70 kDa is not known, and is under investigation. The six isoforms of haptoglobin-1 precursor, exhibited as a chain of protein spots with slightly different molecular mass and different pIs, suggest the presence of post-translational modifications.

These results indicate that quantification of haptoglobin-1 precursor expression in the serum of ovarian cancer patients, for example by ELISA or radio-immunoassay, can be used as a diagnostic marker for screening purposes.

### EXAMPLE 5

### Immunohistochemical Analysis

Paraffin-processed archival tissues were obtained from the Department of Pathology, Royal Women's Hospital, Melbourne. These included normal ovaries (n=6) needed for control comparisons, which were removed from patients undergoing surgery as a result of suspicious ultrasound images, palpable abdominal masses and family history. The pathology diagnosis and tumour grade was determined by two staff pathologists in the Department of Pathology, Royal Women's Hospital, Melbourne. The classification of the tumours was carried out as part of the clinical diagnosis. Histological grading of ovarian carcinoma was performed by the method described by Silverberg (2000).

Tissue sections were cut at 4 μm thickness, mounted on poly-L-lysine coated slides and incubated for 1 h at 60° C. Sections were brought to water through 3 changes each of xylene and ethanol. Antigen unmasking was performed using citrate buffer (pH 6.0) in a microwave oven. Endogenous peroxidases were removed using 3% hydrogen peroxide in methanol, and endogenous biotin activity was blocked using a sequence of diluted egg white (5% in distilled water) and diluted skim milk powder (5% in distilled water). Sections were incubated for lh in anti-haptoglobin monoclonal antibody (Sigma, St Louis USA) diluted 1/10 000 in 1% BSA in Tris buffer (100 mM, pH 7.6). Antibody binding was amplified using biotin and streptavidin HRP (DAKO, Denmark) for 15 min each, and the complex visualized using diaminobenzidine. Nuclei were lightly stained with Mayer's haematoxylin. An isotype IgG1, suitably diluted, was substituted for the antibody as a negative control.

Sections were assessed microscopically for positive diaminobenzidine staining. The intensity of haptoglobin expression was scored in a blind fashion as negative, weak, moderate or strong immunoreactivity. In addition to the type of staining, the tissue and cellular distribution of staining was determined.

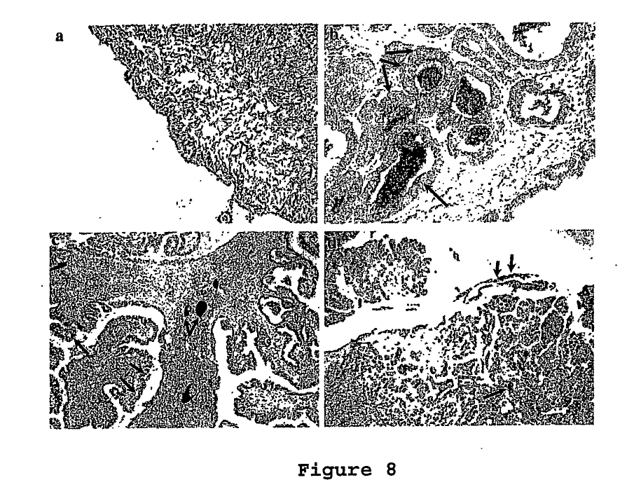

FIG. 8 shows the results of an immunohistochemical comparison between samples of normal ovarian epithelium and of serous and endometrioid ovarian tumours at different grades. No immunoreactive haptoglobin-1 precursor was detected in normal ovarian surface epithelium or stroma, as shown in FIG. 8a. In grade 1 and grade 3 serous and endometrioid ovarian tumours, shown in FIGS. 8b, 8c and 8d respectively, high expression of immunoreactive haptoglobin-1 precursor was detected in epithelium, stroma and ovarian vessels. The staining was mostly cytoplasmic, with the majority of the staining being observed in scattered cell groups. Tumours with a glandular pattern tended to have more staining. Strong staining was evident in areas with myxomatous stroma or vascular spaces, as well as ovarian vessels. Without wishing to be limited by any proposed mechanism, we believe that these results suggest that haptoglobin precursor is expressed by ovarian tumour cells, but that elevated haptoglobin precursor concentrations in the serum of ovarian cancer patients are most probably of hepatic origin.

### EXAMPLE 6

### Expression of Haptoglobin-1 Precursor Pre- And Post-Chemotherapy

The efficacy of detecting the expression of haptoglobin-1 precursor in samples from biological fluid from ovarian cancer patients before and after six cycles of chemotherapy was assessed.

The chemotherapy treatment comprised a conventional combination regimen consisting of carboplatin (AUC 5)/taxol (175 mg/m²body weight) following surgery. The combination drugs were given to patients every three weeks by intravenous infusion. The pre-chemotherapy sample was taken prior to surgery, while the post-chemotherapy sample was taken five months after the therapy was completed. The biological fluid examined was serum. Haptoglobin-1 precursor and CA 125 values were determined in serum samples taken before and after each cycle of therapy.

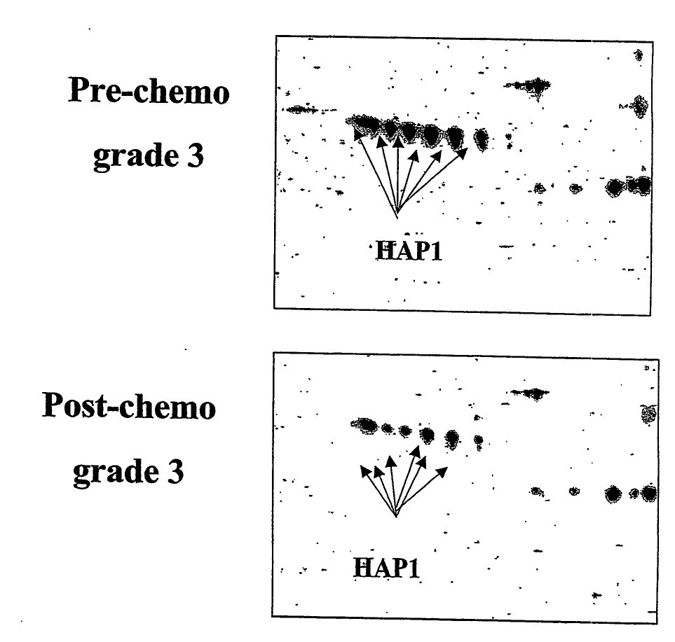

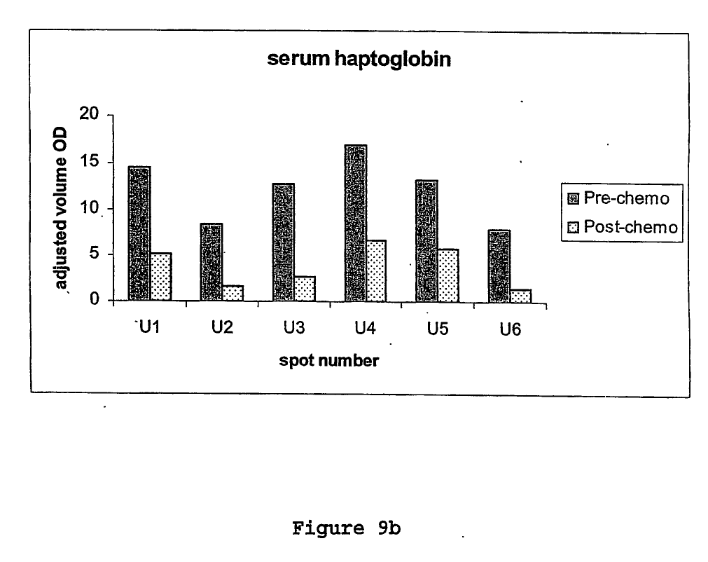

The expression of haptoglobin-1 precursor before and after the chemotherapy is shown in FIGS. 9a and 9b. It can be seen that the level of expression of haptoglobin-1 precursor decreased after chemotherapy relative to the level before chemotherapy.

Before surgery the level of CA 125 in serum from this patient was 376 U/ml. After completion of the chemotherapy the level of CA 125 had reduced to 16 U/ml. Hence the decrease in the level of haptoglobin-1 precursor in the biological fluid after chemotherapy treatment correlated with the decrease in the level of CA 125 in the biological fluid after chemotherapy.

### EXAMPLE 7

### Evaluation of Haptoglobin-1 Precursor Concentration and Its Isoforms in Biological Fluids

The efficacy of ovarian cancer treatment, recurrence of disease following treatment, and the early detection of the onset of ovarian cancer is evaluated by quantifying the concentration of haptoglobin-1 precursor and its isoforms in biological fluids by
(i) direct or indirect sandwich ELISA using either polyclonal or monoclonal antibodies that target intermediate and βchain epitopes
(ii) fluorescent bead-based immunoassasy (Luminx technology) and/or
(iii) magnetic bead-based immunoassay and mass spectrometry analysis.

The assay of haptoglobin-1 precursor is performed in conjunction with the determination of other analytes associated with ovarian cancer using methods known in the art, including but not limited to ELISA assays for CA125 (Mackay and Creasman, 1995), ILK (PCT/AU03/01058), TADG-12 (Underwood L J, 2000), serotransferrin (Kawakami et al, 1999), neutrophil gelatinase associated lipocalin (Kjeldsen et al, 1994), soluble CD163 (Baeton et al, 2003) and Gc-globulin (Jorgensen et al, 2004).

As shown in Table 3, the level of expression of haptoglobin-1 precursor can be used in conjunction with other markers in order to improve the sensitivity and specificity of the test.

TABLE 3
Expression of haptoglobin-1 precursor,
serrotransferin and soluble integrin-linked kinase in the
serum of ovarian cancer patients before and after
chemotherapy

| | | | | | | CA 125 U/ml |
|---|---|---|---|---|---|---|
| | Weeks | | Haptoglobin-1 | | | before |
| Patient | after | | precursor | Serotransferrin | Soluble ILK | after |
| number | treatment | | expression | expression | expression | treatment |
| 1 | 5 | months | Decreases | Increases | Decreases | 370 |
| | | | | | 16 | |
| 2 | 6 | weeks | Increases | Increases | Decreases | 220 |
| | 9 | weeks | Increases | Increases | decreases | 407 |
| | | | further | further | 220 | |
| | | | | | 392 | |

The lack of suppression of haptoglobin-1 precursor in patient 2 after chemotherapy correlates with an increase in CA 125 concentration. This result may indicate the development of resistance to the chemotherapeutic agent, and is being further investigated.

Discussion

Our findings show that serum concentrations of haptoglobin-1 precursor are significantly increased in early stage ovarian cancer patients. Without wishing to limit any proposed mechanism, we believe that enhanced hepatic synthesis of haptoglobin precursor may occur due to an acute phase response in ovarian cancer patients, resulting in elevated concentrations of serum haptoglobin precursor. We have demonstrated a semi-quantitative correlation between the level of haptoglobin precursor expression and the grade of the cancer. We envisage that a quantitative correlation can readily be established using quantitative assay methods such as immunoassay. We consider that haptoglobin-1 precursor represents a new and useful biomarker for ovarian cancer diagnosis. The lack of immunoreactive haptoglobin-1 precursor expression in normal epithelium and the increased expression of immunoreactive haptoglobin-1 precursor in advanced stage tumours suggests that haptoglobin-l precursor is critical for ovarian cancer progression.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed below.

### REFERENCES

Baeton et al, 2003, Arthritis Research and Therapy 5: 90
Beutler, E., Gelbart, T., and Lee, P. Haptoglobin polymorphism and iron homeostasis, Clin Chem. 48: 2232-5., 2002.
Diamandis, E. P., Yousef, G. M., Soosaipillai, A. R., and Bunting, P. (2000) Clin Biochem 33(7), 579-83.
Dobryszycka, W., Katnik-Prastowska, I., Gerber, J., Lemanska, K., Utko, K., and Rozdolski, K. (1999) Arch Immunol Ther Exp (Warsz) 47(4), 229-36.
Hanley, J. M., and Heath, E. C. (1985) Arch Biochem Biophys 239(2), 404-19.
Haugen, T. H., Hanley, J. M., and Heath, E. C. (1981) J Biol Chem 256(3), 1055-7.
Jorgensen et al, 2004 Scandinavian Journal of Clinical and Laboratory Investigation 64:157
Kawakami et al 1999 International Journal of Andrology 22: 224
Kim, J. H., Skates, S. J., Uede, T., Wong, K. K., Schorge, J. O., Feltmate, C. M., Berkowitz, R. S., Cramer, D. W., and Mok, S. C. (2002) JAMA 287(13), 1671-9.
Kjeldsen et al 1994, Blood 83: 799
Luo, L. Y., Bunting, P., Scorilas, A., and Diamandis, E. P. (2001) Clin Chim Acta 306(1-2), 111-8.
Mackay, S. E. and Creasman, W. T. (1995) J. Clin Oncol 13 783-93.
Meechan, K. L., Holland, J. W. and Dawlings. H. J., (2002) Prostate 50(1)54-64.
Misumi, Y., Tanaka, Y., and Ikehara, Y. (1983) Biochem Biophys Res Commun 114(2), 729-36.
Mok, S. C., Chao, J., Skates, S., Wong, K., Yiu, G. K., Muto, M. G., Berkowitz, R. S., and Cramer, D. W. (2001) J Natl Cancer Inst 93(19), 1458-64.
Mueller, W. K., Handschumacher, R., and Wade, M. E. (1971) Obstet Gynecol 38(3), 427-35.
Ono, K. et al., 2000, Identification by cDNA microarray of genes involved in ovarian canrinogenesis, Cancer Research, 60: 5007-5011.
Petricoin, E. F., Ardekani, A. M., Hitt, B. A., Levine, P. J., Fusaro, V. A., Steinberg, S. M., Mills, G. B., Simone, C., Fishman, D. A., Kohn, E. C., and Liotta, L. A. (2002) Lancet 359(9306), 572-7.
Scholler, N., Fu, N., Yang, Y., Ye, Z., Goodman, G. E., Hellstrom, K. E., and Hellstrom, I. (1999) Proc Natl Acad Sci USA 96(20), 11531-6.
Schreiber, G., and Urban, J. (1978) Rev Physiol Biochem Pharmacol 82, 27-95.
Schummer, M., Ng, W. V., Bumgarner, R. E., Nelson, P. S., Schummer, B., Bednarski, D. W., Hassell, L., Baldwin, R. L., Karlan, B. Y., and Hood, L. (1999) Gene 238(2), 375 85.
Silverberg, S (2000) Int. J. Gynecol. Pathol 19 7-15.
Thompson, S., Cantwell, B. M., Matta, K. L., and Turner, G. A. (1992) Cancer Lett 65(2), 115-21.
Thompson, S., Dargan, E., and Turner, G. A. (1992) Cancer Lett 66(1), 43-8.
Underwood, L. J., Shigemasa, K., Tanimoto, H, Beard, J. B., Schneider, E. N., Wang, Y., Parmley, T. H., O'Brien, T. J., Biochim Biophys Acta. (2000) 1502(3):337-50
Wassell, J. (2000) Clin Lab 46(11-12), 547-52.
Wilson, N. L., Schulz, B. L., Karlsson, N. G., and Packer, N. H. (2002) J Proteome Res 1(6), 521-9.

1-16. (canceled)

17. A method of detecting ovarian cancer comprising;
determining the concentration of haptoglobin-1 precursor in a sample of a biological fluid from a subject suspected to be suffering from ovarian cancer, wherein an increased concentration of haptoglobin-I precursor compared to the concentration of haptoglobin-1 precursor in a control sample is an indication of the presence of said cancer.

18. The method of claim 17, wherein said biological fluid is selected from the group consisting of blood, plasma, serum, ascitic fluid and urine.

19. The method of claim 17, wherein the biological fluid has been subjected to a preliminary step to deplete high abundance proteins, wherein said preliminary step increases the sensitivity of detection of low abundance proteins.

20. The method of claim 17, further comprising the step of correlating the level of haptoglobin-1 precursor with one or more other ovarian cancer markers.

21. The method of claim 20, wherein said ovarian cancer marker is selected from the group consisting of integrin-linked kinase (ILK), CA125, TADG-12, mesothelin, kallikrein 10, prostasin, osteopontin, creatine kinase β, serotransferrin, neutrophil-gelatinase associated lipocalin (NGAL), CD163, and Gc-globulin.

22. The method of claim 17, wherein said step of determining the concentration of haptoglobin-1 precursor is by a probe specific for haptoglobin-1 precursor selected from the group consisting of an antibody and a nucleic acid.

23. The method of claim 22, wherein said antibody is selected from the group consisting of a monoclonal antibody, an antibody that does not react with an epitope within the alpha chain and an antibody specific for a haptoglobin-1 precursor.

24. The method of monitoring the efficacy of treatment of ovarian cancer, comprising;
determining the concentration of haptoglobin-1 precursor in a sample of a biological fluid from a subject suspected to be suffering from ovarian cancer, wherein a decrease in haptoglobin-1 precursor level compared to the level before treatment is an indication of efficacy of said treatment.

25. The method of claim 24, wherein said biological fluid is selected from the group consisting of blood, plasma, serum, ascitic fluid and urine.

26. The method of claim 24, wherein the biological fluid has been subjected to a preliminary step to deplete high abundance proteins, wherein said preliminary step increases the sensitivity of detection of low abundance proteins.

27. The method of claim 24, further comprising the step of correlating the level of haptoglobin-1 precursor with one or more other ovarian cancer markers.

28. The method of claim 27, wherein said ovarian cancer marker is selected from the group consisting of integrin-linked kinase (ILK), CA125, TADG-12, mesothelin, kallikrein 10, prostasin, osteopontin, creatine kinase β, serotransferrin, neutrophil-gelatinase associated lipocalin (NGAL), CD163, and Gc-globulin.

29. The method of claim 24, wherein said step of determining the concentration of haptoglobin-1 precursor is by a probe specific for haptoglobin-1 precursor selected from the group consisting of an antibody and a nucleic acid.

30. The method of claim 29, wherein said antibody is selected from the group consisting of a monoclonal antibody, an antibody that does not react with an epitope within the alpha chain and an antibody specific for a haptoglobin-1 precursor.

31. A method of assessing the severity of ovarian cancer comprising;
determining the concentration of haptoglobin-1 precursor in a biological fluid of a subject diagnosed with, or suspected to be suffering from, ovarian cancer, wherein an increased concentration of haptoglobin-1 precursor compared to the concentration of haptoglobin-1 precursor in a control sample is an indication of the severity of said cancer.

32. The method of claim 31, wherein said biological fluid is selected from the group consisting of blood, plasma, serum, ascitic fluid and urine.

33. The method of claim 31, wherein the biological fluid has been subjected to a preliminary step to deplete high abundance proteins, wherein said preliminary step increases the sensitivity of detection of low abundance proteins.

34. The method of claim 31, further comprising the step of correlating the level of haptoglobin-1 precursor with one or more other ovarian cancer markers.

35. The method of claim 34, wherein said ovarian cancer marker is selected from the group consisting of integrin-linked kinase (ILK), CA125, TADG-12, mesothelin, kallikrein 10, prostasin, osteopontin, creatine kinase β, serotransferrin, neutrophil-gelatinase associated lipocalin (NGAL), CD163, and Gc-globulin.

36. The method of claim 31, wherein said step of determining the concentration of haptoglobin-1 precursor is by a probe specific for haptoglobin-1 precursor selected from the group consisting of an antibody and a nucleic acid.

37. The method of claim 36, wherein said antibody is selected from the group consisting of a monoclonal antibody, an antibody that does not react with an epitope within the alpha chain and an antibody specific for a haptoglobin-1 precursor.

38. A kit comprising;
a probe specific for haptoglobin-1 precursor selected from the group consisting of an antibody and nucleic acid; and
instructions.

39. A kit according to claim 38, wherein said antibody is selected from the group consisting of a monoclonal antibody, an antibody that does not react with an epitope within the alpha chain and an antibody specific for a haptoglobin-1 precursor.
